(19) United States
(12) Patent Application Publication
(10) Pub. No.: US 2002/0025535 A1
(43) Pub. Date: Feb. 28, 2002

(54) Prioritization of combinatorial library screening

(76) Inventors: David J. Diller, East Windsor, NJ (US); Kenneth M. Merz JR., Yardley, PA (US)

Correspondence Address: HESLIN ROTHENBERG FARLEY & MESITI PC, 5 COLUMBIA CIRCLE, ALBANY, NY, 12203, US

(21) Appl. No.: 09/832,786

(22) Filed: Apr. 11, 2001

Related U.S. Application Data
(63) Continuation-in-part of application No. 09/595,096, filed on Jun. 15, 2000.

(51) Int. Cl. G01N 33/53; G06F 19/00; G01N 33/48; G01N 33/50

(52) U.S. Cl. 435/7.1; 702/19

(57) ABSTRACT

In a computational method of assessing a combinatorial library for complementarity to a target molecule, ligands from the library are first docked to the target molecule. The docking results are clustered using as a metric the rms deviation between the core of two docked molecules. The library is rated as to complementarity to the target molecule according to the relative number of ligands in the top cluster.

### CROSS-REFERENCE TO RELATED APPLICATIONS

This application is a continuation-in-part of copending U.S. application Ser. No. 09/595,096, filed on Jun. 15, 2000, which is incorporated herein by reference in its entirety.

### FIELD OF THE INVENTION

The present application relates to computational methods for prioritizing selection of combinatorial libraries for screening, using high throughput molecular docking techniques.

### BACKGROUND OF THE INVENTION

With the advent of combinatorial chemistry and the resulting ability to synthesis large collections of compounds for a broad range of targets, it has become apparent that the capability to effectively prioritize screening efforts is crucial to the rapid identification of the appropriate region of chemical space for a given target. Given the power of combinatorial chemistry and high throughput screening, it is no longer necessary to exclusively use rational design tools to generate lead compounds. With the volume of chemistry space now synthetically accessible, however, it is impossible to adequately sample all potential compounds, so even with the combinatorial chemistry paradigm, some “rational” decision making is required. For example, it is important to rapidly focus on the correct regions of chemistry space (as defined using physical properties like solubility, shape, intestinal absorption, and other properties). Effective prioritization tools would allow scientists to obtain leads in a cost effective and efficient manner, and also to test virtual libraries against novel targets prior to active synthesis and bioanalysis, thereby, reducing costs. In addition, with the coming explosion of targets expected from the complete sequencing of the human genome and the many genomes to follow, it is imperative that resources not be wasted screening areas of chemistry space unlikely to yield active compounds. The new challenge arising with the advent of combinatorial chemistry, then, is prioritizing this election of combinatorial libraries.

Co-pending U.S. application, Ser. No. 09/595,096, describes a molecular docking method for prioritizing screening efforts. Using this method, individual compounds of a library or collection are docked to the target and ranked by a scoring function. A high-ranking subset of the compound, rather than the entire library, may then be assayed for activity. While this method has proven useful for guiding selection of individual compounds for testing, there remains a need for a way to prioritize combinatorial library screening efforts, that is, rather than ranking individual compounds, combinatorial libraries of compounds are ranked.

### SUMMARY OF THE INVENTION

A method for addressing overall library complementary to the target, and so, for prioritizing combinatorial libraries for screening against targets has now been developed. The method utilizes the significant amount of information extractable from trends in high throughput docking data rather than from individual compounds, identifying actual or virtual libraries likely to possess activity without the necessity for actual synthesis and bioanalysis.

In one aspect, the present invention relates to a method of assessing a combinatorial library for complementarity to a target molecule. The library comprises a plurality of ligands having a common core. The method comprises docking each ligand of the plurality of ligands to the target molecule to generate a plurality of ligand positions relative to the target molecule in a plurality of ligand-target complex formations, the plurality of ligand positions comprising a plurality of common core positions relative to the target molecule; determining rms deviation of each common core position of the plurality of common core positions from other common core positions; and forming clusters according the rms deviation.

In another aspect, the present invention relates to a system for assessing a combinatorial library for complementarity to a target having at least one binding site, the combinatorial library comprising a plurality of ligands, each based on a common core. The system includes means for docking each ligand of the plurality of ligands to the target molecule to generate a plurality of ligand positions relative to the target molecule in a plurality of ligand-target molecule complex formations, the plurality of ligand positions comprising a plurality of common core positions relative to the target molecule; means for determining an rms deviation of each common core position of the plurality of common core positions from other common core positions; and means for forming clusters according to the rms deviation.

In yet another aspect, the present invention relates to at least one program storage device readable by a machine, tangibly embodying at least one program of instructions executable by the machine to perform a method for assessing a combinatorial library for complementarity to a target having at least one binding site, the combinatorial library comprising a plurality of ligands, each based on a common core. The method includes docking each ligand of the plurality of ligands to the target molecule to generate a plurality of ligand positions relative to the target molecule in a plurality of ligand-target molecule complex formations, the plurality of ligand positions comprising a plurality of common core positions relative to the target molecule; determining an rms deviation of each common core position of the plurality of common core positions from other common core positions; and forming clusters according to the rms deviation.

### BRIEF DESCRIPTION OF THE DRAWINGS

The above-described objects, advantages and features of the present invention, as well as others, will be more readily understood from the following detailed description of certain preferred embodiments of the invention, when considered in conjunction with the accompanying drawings in which:

FIGS. 1A-1C conceptually depict protein-ligand complex formation;

FIG. 2 is a flowchart of one embodiment of a molecular docking approach in accordance with the principles of the present invention;

FIG. 3 is a flowchart of one embodiment of a molecular conformational search procedure which can be employed by the docking approach of FIG. 2, in accordance with the principles of the present invention;

FIG. 4 is a flowchart of one embodiment of establishing a binding site image for use with the molecular docking approach of FIG. 2, in accordance with the principles of the present invention;

FIG. 5 is a flowchart of one embodiment of a matching procedure for use with the molecular docking approach of FIG. 2, in accordance with the principles of the present invention;

FIG. 6 is a flowchart of one embodiment of an optimization stage for optimizing ligand positions within identified matches for use with the molecular docking approach of FIG. 2, in accordance with the principles of the present invention;

FIG. 7 graphically depicts a hydrogen bonding potential and a steric potential for use in atom pairwise scoring in accordance with the principles of the present invention;

FIG. 8 depicts one embodiment of a computer environment providing and/or using the capabilities of the present invention;

FIG. 9 is a conceptual representation of the binding site of a target protein having pockets P1, P2 and P3, with compound from a combinatorial library positioned within the binding center;

FIG. 10 is a graph showing cluster sizes for compounds from combinatorial library PL 792 docked to the target protein, plasmepsin II from Plasmodium falciparum;

### DETAILED DESCRIPTION OF THE INVENTION

The present invention relates to a method of assessing a combinatorial library for complementarity to a target molecule. In the method, each ligand in the library is docked to the target molecule to generate a ligand position relative to the target. For each ligand, the rms deviation of the position of the common core of each ligand from the position of the common core of other ligands in the library is then determined. Finally, the data is organized via cluster analysis, wherein clusters are formed according to the rms deviation between common cores of the ligands, and the library is ranked according to the relative number of ligands in the top cluster.

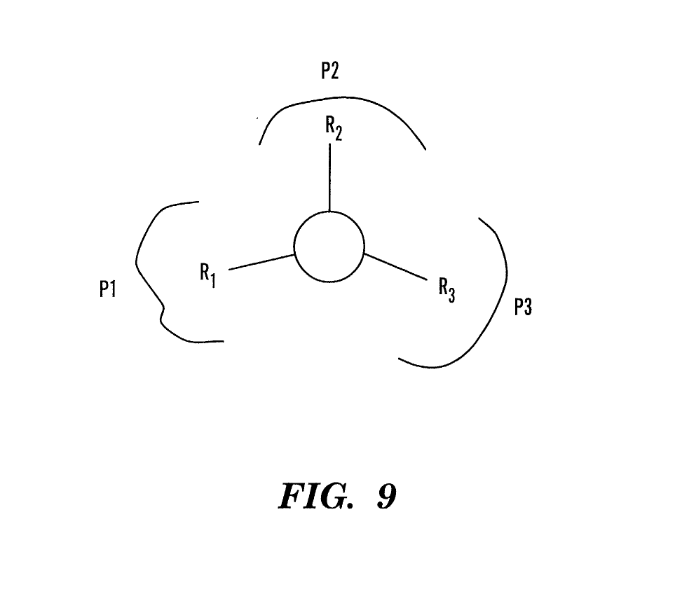

The combinatorial libraries that may be screened using the method of the present invention generally contain thousands of compounds that potentially bind to the target and are thus termed “ligands”. These libraries are constructed around a basic chemical structure which is varied by substituents attached at a limited number of positions. The basic chemical structure is referred to as the “common core” for purposes of the present invention. For example, the common core of an aspartyl protease inhibitor library is shown in FIG. 9. A large number of different synthons may be substituted at predetermined positions, yielding a library containing from tens of thousands to millions of compounds. For example, in the structure of FIG. 9, R₁, R₂, R₃ and R₄ indicate locations where the various synthons may be substituted.

The target molecule may be any biochemical that can bind to ligands in the library, especially, proteins and nucleotides. The method of the present invention is particularly intended for use with proteins, and especially, proteins for which structural data, generally crystallographic data, is available. Potential binding sites are typically identified in the structure by visual inspection.

In the method of the present invention, each ligand is docked to the target molecule. The docking procedure generates at least one position for each ligand relative to the target molecule wherein the ligand is matched to complementary binding spots on the target. A preferred docking procedure includes the following steps: performing a pre-docking conformational search to generate multiple solution conformations of each ligand; generating a binding site image of the target molecule; matching hot spots of the binding site image to atoms in at least one solution conformation of the multiple solution conformations of each ligand to obtain at least one ligand position relative to the target molecule; and optimizing the ligand position while allowing translation, orientation, and rotatable bonds of the ligand to vary, and while holding the target molecule fixed.

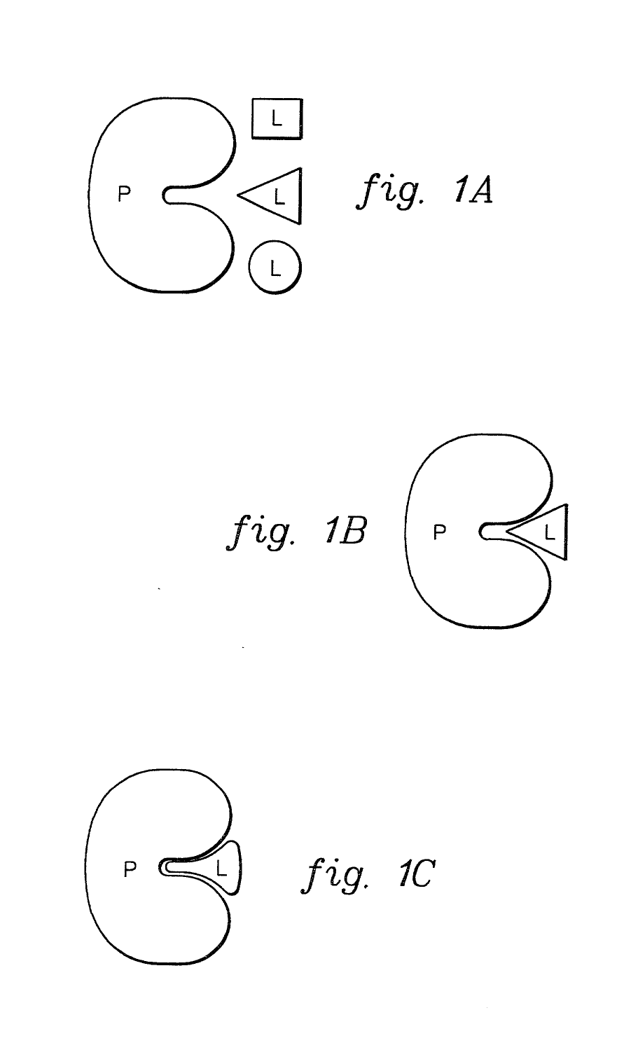

The docking procedure is based on a conceptual picture of protein-ligand complex formation (see FIGS. 1A-1C). Initially, the ligand (L) adopts many conformations in solution. The protein (P) recognizes one or several of these conformations. Upon recognition, the ligand, protein and solvent follow the local energy landscape to form the final complex. While the procedure is described in terms of a protein target, the same steps may be performed when the target is a biomolecule other than a protein, such as a nucleotide.

This simple picture of target molecule/ligand complex formation is converted into an efficient computational model, as follows. The initial solution conformations are generated using a straightforward conformational search procedure. One might view the conformational search part of this technique as part of the entire docking process, but since it involves only the ligand, it can be decoupled from the purely docking steps. This is justified since 3-D databases of conformations for a collection of molecules can readily be generated and stored for use in numerous docking studies (for example, using Catalyst, see A. Smellie, S. D. Kahn, S. L. Teig, “Analysis of Conformational Coverage. 1. Validation and Estimation of Coverage”, J. Chem. Inf. Comput. Sci. (1995) v235, pp285-294; and A. Smellie, S. D. Kahn, S. L. Teig, “Analysis of Conformational Coverage. 2. Application of Conformational Models”, J. Chem. Inf. Comput. Sci. (1995) v235, pp295-304). The recognition stage is modeled by matching atoms of the ligand to interaction with “hot spots” in the binding site. The final complex formation is modeled using a gradient based optimization technique with a simple energy function. During this final stage, the translation, orientation, and rotatable bonds of the ligand are allowed to vary, while the target molecule and solvent are held fixed.

Most docking methods can be classified into one of two loosely defined categories: (1) stochastic, such as AutoDock, (Goodford, P. J., “A Computational Procedure for Determining Energetically Favorable Binding Sites on Biologically Important Macromolecules,” Journal of Medicinal Chemistry, 1985, Vol. 28(7), p. 849-857; Goodsell, D. S. and A. J. Olson, “Automated Docking of Substrates to Proteins by Simulated Annealing,” PROTEINS: Structure, Function and Genetics, 1990, Vol. 8, p. 195-202), GOLD (Jones, G., et al., “Development and Validation of a Generic Algorithm for Flexible Docking,” Journal of Molecular Biology, 1997, Vol. 267, p. 727-748), TABU (Westhead, D. R., D. E. Clark, and C. W. Murray, “A Comparison of Heuristic Search Algorithms for Molecular Docking,” Journal of Computer-Aided Molecular Design, 1997, Vol. 11, p. 209-228; and Baxter, C. A. et al., “Flexible Docking Using Tabu Search and an Empirical Estimate of Binding Affinity,” PROTEINS: Structure, Function, and Genetics, 1998, Vol. 33, p. 367-382), and Stochastic Approximation with Smoothing (SAS) (Diller, D. J. and C. L. M. J. Verlinde, “A Critical Evaluation of Several Global Optimization Algorithms for the Purpose of Molecular Docking,” Journal of Computational Chemistry, 1999, Vol. 20(16), p. h1740-1751); or (2) combinatorial, for example, DOCK (Kuntz, I. D., et al., “A Geometric Approach to Macromolecular-ligand Interactions,” Journal of Molecular Biology, 1982, Vol. 161, p. 269-288); Kuntz, I. D., “Structure-based Strategies for Drug Design and Discovery,” Science, 1992, Vol. 257, p. 1078-1082; Makino, S. and I. D. Kuntz, “Automated Flexible Ligand Docking Method and Its Application for Database Search,” Journal of Occupational Chemistry, 1997, Vol. 18(4), p. 1812-1825), FlexX (Rarey, M., et al., “A Fast Flexible Docking Method Using an Incremental Construction Algorithm,” Journal of Molecular Biology, 1996, Vol. 261, p. 470-489; Rarey, M., B. Kramer, and T. Lengauer, “The Particle Concept: Placing Discrete Water Molecules During Protein-ligand Docking Predictions,” PROTEINS: Structure, Function, and Genetics, 1999, Vol. 34, p. 17-28; Rarey M., B. Kramer, and T. Lengauer, “Docking of Hydrophobic Ligands With Interaction-based Matching Algorithms,” Bioinformatics, 1999, Vol. 15(3), p. 243-250), and HammerHead (Welch, W., J. Ruppert, and A. N. Jain, “Hammerhead: Fast Fully Automated Docking of Flexible Ligands to Protein Binding Sites,” Chemistry & Biology, 1996, Vol. 3(6), p. 449-462).

The stochastic methods, while often providing more accurate results, are typically too slow to search large databases. The method presented herein falls into the combinatorial group. This approach is analogous to FlexX and HammerHead in that it attempts to match interactions between the ligand and receptor. It differs from these and most other docking techniques significantly in how it handles the flexibility of the ligand. Most current combinatorial docking techniques handle flexibility using an incremental construction approach, whereas the technique described herein uses an initial conformational search followed by a gradient based minimization in the presence of the target.

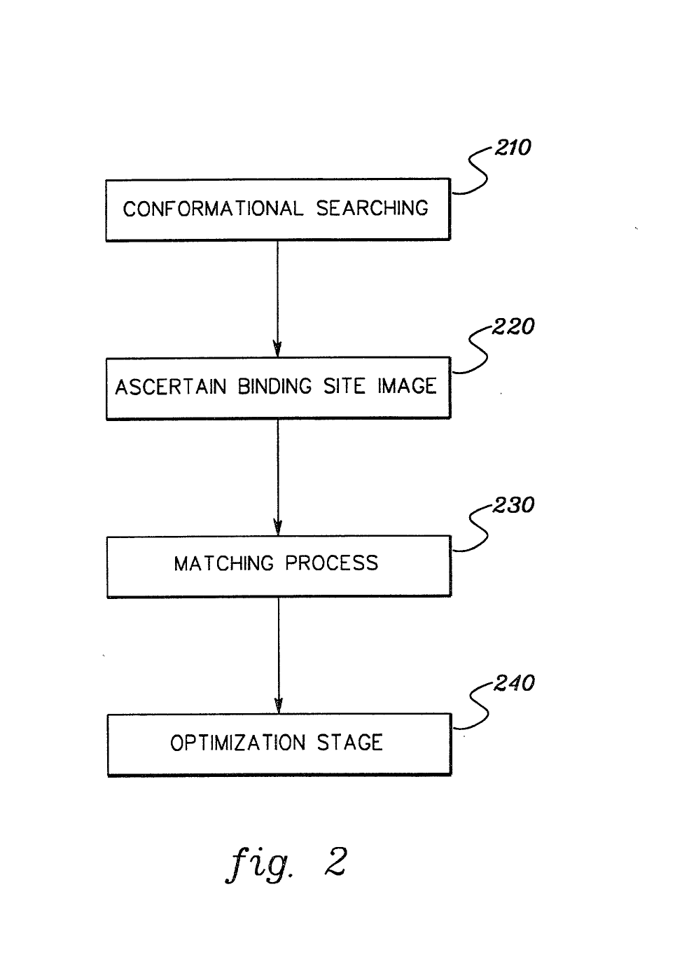

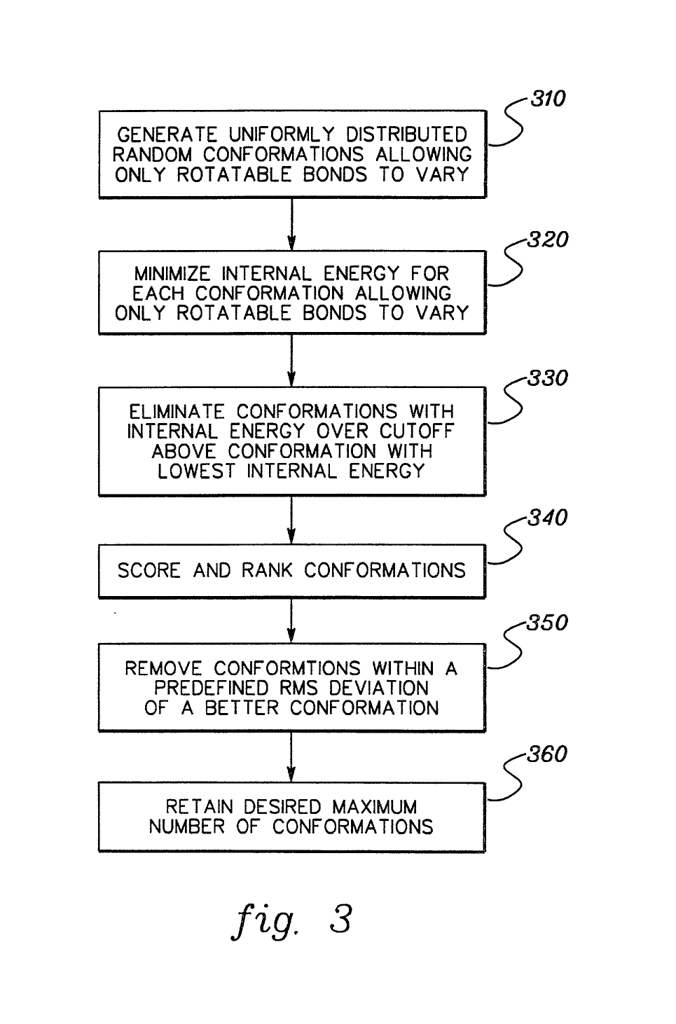

A generalized technique is depicted in FIG. 2. Initially, a conformational search procedure 210 is performed for an entire library or collection, with the resulting conformations stored for future use. A binding site image is then created using the target molecule structure 220. A matching procedure is performed to form an initial complex by initially positioning a given conformation of a ligand as a rigid body into the binding site 230. Finally, a flexible optimization is performed wherein the matches are pruned and then optimized to attain the final result 240. Each of these steps of a docking approach is described in greater detail below with reference to FIGS. 3-6, respectively.

A straightforward yet effective conformational search procedure is preferred. A conformational search is performed once for an entire library or a collection, with the resulting conformations stored for future use. If desired, the conformational searching can be periodically repeated.

Referring to FIG. 3, uniformly distributed random ligand conformations are generated allowing only rotatable bonds to vary 310. For example, 1,000 uniformly distributed random conformations can be generated varying only the rotatable bonds. The internal energy of each conformation is then minimized, again allowing only rotatable bonds to vary 320. Internal energy can be estimated, for example, using van der Waals potentials and dihedral angle term, reference: Diller, D. J. and C. L. M. J. Verlinde “A Critical Evaluation of Several Global Optimization Algorithms for the Purpose of Molecular Docking,” Journal of Computational Chemistry, 1999, Vol. 20(16), p. 1740-1751, which is hereby incorporated herein by reference in its entirety. Each conformation can be minimized using, for example, a BFGS (Broyden-Fletcher-Goldfarb-Shanno) optimization algorithm, e.g., reference Press, W. H., et al., Numerical Recipes in C, 2 ed., 1997, Cambridge: Cambridge University Press. 994, which is hereby incorporated herein by reference in its entirety.

Conformations with internal energy over a selected cut-off above a conformation with the lowest internal energy are eliminated 330. For example, any conformation with an internal energy of 15 kcal/mol above the conformation with the lowest internal energy is eliminated. The remaining conformations are scored and ranked 340. Conformations can be ranked by a score defined as:

Score=Strain−0.1×SASA

where SASA is the “solvent accessible surface area” of a particular conformation; and “strain” of a given conformation of a given molecule is the internal energy of the given conformation minus the internal energy of the conformation of the given molecule with the lowest internal energy. Conformations within a pre-defined rms deviation of a better conformation are removed 350. For example, any conformation within an rms deviation of 1.0 Å of a higher ranked (i.e., better) conformation can be removed. This clustering is a means to remove redundant conformations. A maximum number of desired conformations, for example, 50 conformations, are retained at the end of the conformational analysis step 360.

If more than the desired number of conformations remain after clustering, then the lowest ranked conformations can be removed until the desired number of conformations remain.

The process of a small molecule binding to a target is a balance between “salvation” by water versus “solvation” by the target molecule. With this in mind, the solvent accessible surface area term can be chosen in analogy with simple aqueous salvation models, e.g., reference Eisenberg, D. and A. D. McLachlan, “Solvation Energy in Protein Folding and Binding,” Nature, 1986, Vol. 319, p. 199-203; Ooi, T., et al., “Accessible Surface Areas as a Measure of the Thermodynamic Parameters of Hydration of Peptides,” Proceedings of the National Academy of Sciences, 1987, Vol. 84, p. 3086-3090; and Vajda, S., et al., “Effect of Conformational Flexibility and Solvation on Receptor-ligand Binding Free Energies,” Biochemistry, 1994, Vol. 33, p. 13977-13988, each of which is hereby incorporated herein by reference in its entirety. The key difference in protein versus water “salvation” is that water competes for polar interactions only, while a protein effectively competes for both polar and hydrophobic interactions. Therefore, for purposes of this invention, polar and apolar surface areas are treated identically. The choice of 0.1 as a weight for the surface area term is somewhat arbitrary, but is comparable to the weights chosen for surface area based salvation models. Ultimately, conformations with more solvent accessible surface area are going to be able to interact more extensively with a target and can, therefore, be of somewhat higher strain and still bind tightly. A more refined ranking system could be used with the present invention, but this approach to ranking conformations supplies reasonable conformations.

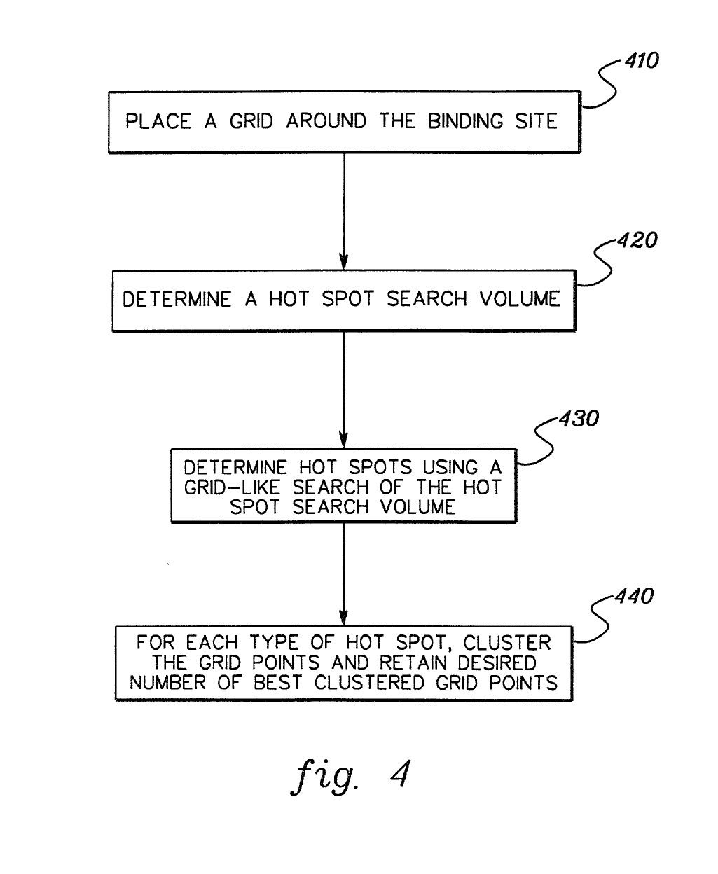

The binding site image comprises a list of apolar hot spots, i.e., points in the binding site that are favorable for an apolar atom to bind, and a list of polar hot spots, i.e., points in the binding site that are favorable for a hydrogen bond donor or acceptor to bind. One procedure for creating these two lists is depicted in FIG. 4. First, in order to find the binding site, a grid is placed around the binding site 410. By way of example, the grid may be at least 20 Å×20 Å×20 Å with at least 5 Å of extra space in each direction. A 0.2 Å spacing can be used for the grid. Next, a “hot spot search volume” is determined 420. This is accomplished by eliminating any grid point inside the target molecule. Any point contained in, for example, a 6.0 Å or larger sphere not touching the target molecule can also be eliminated. The largest remaining connected piece becomes the “hot spot search volume”.

The hot spots can then be determined using a grid-like search of the hot spot search volume 430. By way of example, a grid-like search is described in Goodford, P. J., “A Computational Procedure for Determining Energetically Favorable Binding Sites on Biologically Important Macromolecules,” Journal of Medicinal Chemistry, 1985, Vol. 28(7), p. 849-857, which is hereby incorporated herein by reference in its entirety. To find the apolar hot spots, an apolar probe is placed at each grid point in the hot spot search volume, the probe score is calculated and stored. The process is repeated for polar hot spots. For each type of hot spot, the grid points are clustered and a desired number of best clustered grid points is maintained 440. For example, the top 30 clustered grid points may be retained.

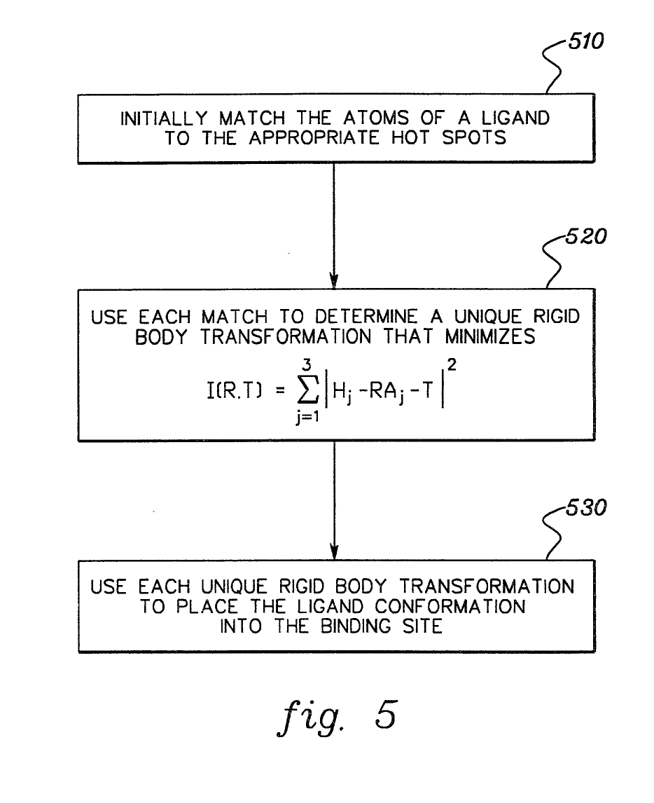

Referring to FIG. 5, in order to initially position a given conformation of a ligand as a rigid body into the binding site, the atoms of the ligand are matched to the appropriate hot spots 510. More precisely, in one example, a triplet of atoms, A₁, A₂, A₃ is considered a match to a triplet of hot spots, H₁, H₂, H₃ if:

i. The type of Aⱼ matches the type of Hⱼ for each j=1,2,3, that is, apolar hot spots match apolar atoms and polar hot spots match polar atoms.

ii. D(Aⱼ, Aₖ)=D(Hⱼ, Hₖ)+δ for all j,k=1,2,3 where D(Aⱼ, Aₖ)and D(Hⱼ, Hₖ) are the distance from Aⱼ to Aₖ and Hⱼ to Hₖ, respectively, and δ is some allowable amount of error, e.g., between 0.25 Å and 0.5 Å.

To restate, a match occurs, in one example, when three hot spots forming a triangle and three atoms of the ligand forming a triangle substantially match. That is, a match occurs when the triangles are sufficiently similar with the vertices of each triangle being the same type and the corresponding edges of similar length. The matching algorithm finds all matches between atoms of a given conformation and the hot spots. Each match then determines a unique rigid body transformation. The rigid body transformation is then used to bring the conformation into the binding site to form the initial target molecule-ligand complex.

In step 520, each match determines a unique rigid body transformation that minimizes

$$I ( R , T ) = {\sum }_{J = 1}^{3} {\vert {H}_{j} - {RA}_{j} - T \vert}^{2}$$

where R is, for instance, a 3×3 rotation matrix and T is a translation vector. Again, a rigid body transformation comprises in one example, a 3×3 rotation matrix, R, and translation vector T, so that points X (the position of an atom of the conformation) are transformed by RX+T. Each rigid body transformation, which can be determined analytically, is then used to place the ligand conformation into the binding site 530. For this aspect of the calculation, several algorithms for finding all matches were tested. The geometric hashing algorithm developed for FlexX (see: Rarey, M., S. Welfing, and T. Lengauer, “Placement of Medium-sized Molecular Fragments Into Active Sites of Proteins,” Journal of Computer-Aided Molecular Design, 1996, Vol. 10, p. 41-54, which is hereby incorporated herein by reference in its entirety), proved to be the most efficient.

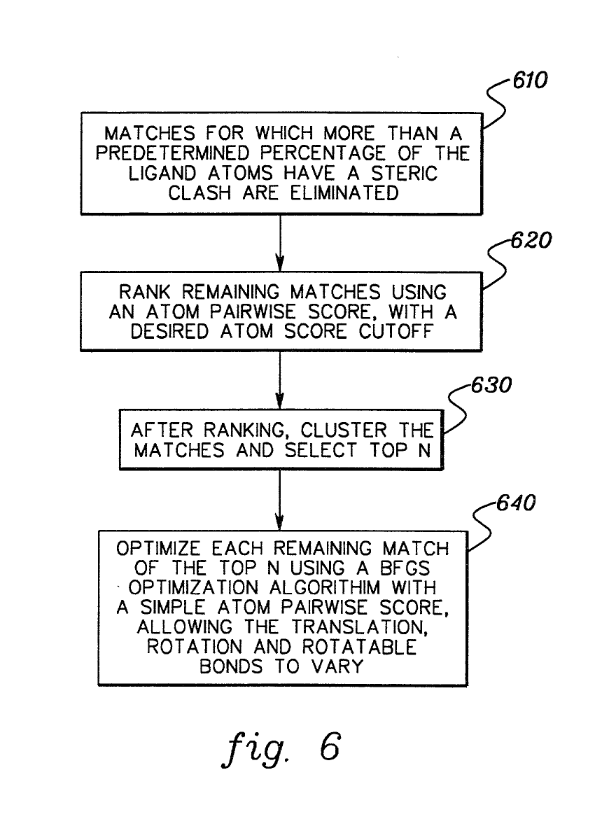

A single ligand conformation can produce up to 10,000 matches with binding hot spots. In the interest of efficiency, most of these matches cannot be optimized, so a pruning/scoring strategy is desired. FIG. 6 depicts one such strategy.

Referring to FIG. 6, initially all matches for which more than a predetermined percentage (e.g., 10%) of the ligand atoms have a steric clash can be eliminated 610. The remaining matches are ranked using an atom pairwise score described below, with an atom score cutoff of for example 1.0-620. Use of a cutoff allows matches that fit reasonably well with a few steric clashes to survive to the final round, and the choice of 1.0 is merely exemplary. After being ranked, the matches are clustered, and the top N matches are selected to move into the final stage 630, where N may comprise, for instance, a number in the range of 25-100.

Each remaining match is optimized using a BFGS optimization algorithm with a simple atom pairwise score 640. In one embodiment, the score can be modeled after the Piecewise Linear Potential (see, Gehlhaar, D. K., et al., “Molecular Recognition of The Inhibitor AG-1343 By HIV-1 Protease: Conformationally Flexible Docking by Evolutionary Programming,” Chemistry & Biology, 1995, Vol. 2, p. 317-324, which is hereby incorporated herein by reference in its entirety) with a difference being that the score used herein is preferably differentiable. For this score, all hydrogens are ignored, and all non-hydrogen atoms are classified into one of four categories:

i. Apolar—anything that cannot form a hydrogen bond.

ii. Acceptor—any atom that can act as a hydrogen bond acceptor, but not as a donor.

iii. Donor—any atom that can act as a hydrogen bond donor, but not as an acceptor.

iv. Donor/Acceptor—any atom that can act as both a hydrogen bond donor and an acceptor.

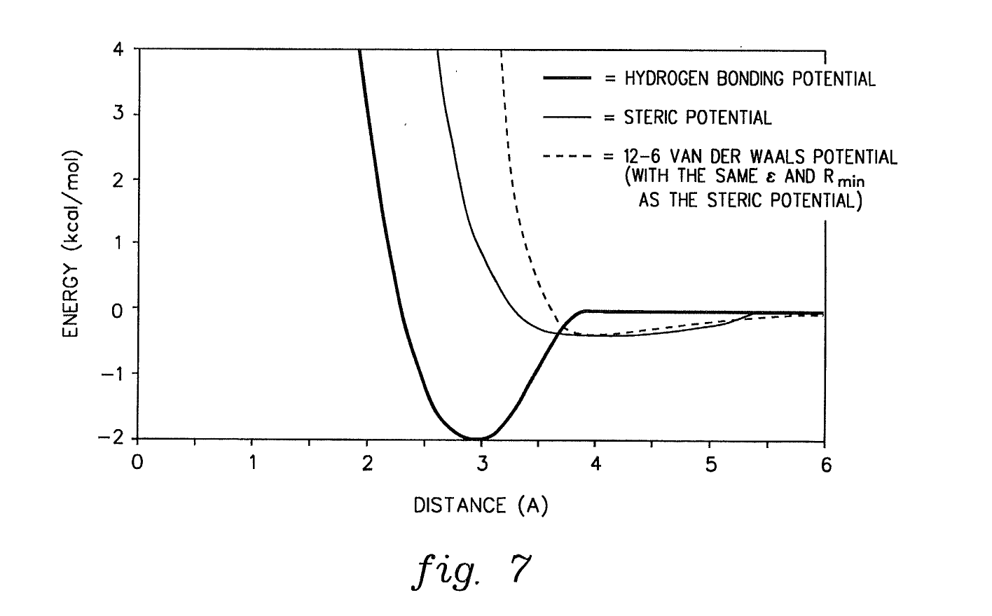

The score between two atoms is calculated using either a hydrogen bonding potential or a steric potential. The two potentials, shown in FIG. 7, have the mathematical form

$$F ( r ) = ϵ [ {( \frac{( 1 + σ ) {R}_{min}^{2}}{{r}^{2} + σ {R}_{min}^{2}} )}^{2} - 2 {( \frac{( 1 + σ ) {R}_{min}^{2}}{{r}^{2} + σ {R}_{min}^{2}} )}^{3} ] Φ ( {r}^{2} : {r}_{1}^{2} , {r}_{0}^{2} )$$

where Rₘᵢₙ is the position of the score minimum, ε is the depth of the minimum, σ is a softening factor, and φ(r:r₁, r₀)is a differentiable cutoff function of r (the distance between the pair of atoms) having the properties that when r<r₁ φ=1 and when r>r₀ φ=0. Each potential, steric and hydrogen bonding, is assigned its own set of parameters. The parameters for these potentials can be chosen by one skilled in the art via intuition and subsequent testing, but they do not need to be fully optimized. Table 2 contains example parameters for the pairwise potentials.

TABLE 2

| | hydrogen bonding | Steric |
|---|---|---|
| | potential | Potential |
| ε | 2.0 | 0.4 |
| σ | 0.5 | 1.5 |
| Rₘᵢₙ | 3.0Å | 4.05Å |
| r₁ | 3.0Å | 5.0Å |
| r₀ | 4.0Å | 6.0Å |

These potentials are very similar to the 12-6 van der Waals potentials used in many force fields, with two differences. First, the softening factor, σ, makes the potentials significantly softer than the typical 12-6 van der Waals potentials (see FIG. 7), i.e., mild steric clashes common in docking runs are tolerated by this potential. In spirit, the softening factor implicitly models small induced fit effects of the target molecule which can be important (see, Murray, C. W., C. A. Baxter, and D. Frenkel, “The Sensitivity of The Results of Molecular Docking to Induced Fit Effects: Application to Thrombin, Thermolysin and Neuraminidase,” Journal of Computer-Aided Molecular Design, 1999, Vol. 12, p. 547-562, which is hereby incorporated herein by reference in its entirety), and in practice, makes the potential much more error tolerant. The second difference is the cutoff function. This function guarantees that the potential is zero beyond a finite distance usually between 5.0 Å and 6.0 Å. This along with some organization of the target molecule atoms significantly speeds up the direct calculation of the score.

An attempt was made to calculate the scores both directly and through precalculated grids. The advantage of using the grids is that the score can be calculated very rapidly. Grids were found to be 5-10 times faster than the direct calculation. The advantage of the direct calculation is that effects, such as target molecule flexibility and solvent mobility, can be accommodated more easily. Since using the grids did not seem to cause any deterioration in the quality of the docking results and since target molecule flexibility or solvent mobility is currently not included, for the results presented hereinbelow, the scores were calculated through precalculated grids. For the purpose of the BFGS optimization algorithm, all derivatives were calculated analytically including those with respect to the rotatable bonds (see, Haug, E. J. and M. K. McCullough, “A Variational-Vector Calculus Approach to Machine Dynamics,” Journal of Mechanisms, Transmissions, and Automation in Design, 1986, Vol. 108, p. 25-30, which is hereby incorporated herein by reference in its entirety).

To test the docking procedure, the GOLD test set was used (see Jones, G., et al., “Development and Validation of a Generic Algorithm for Flexible Docking,” Journal of Molecular Biology, 1997, Vol. 267, p. 727-748, which is hereby incorporated herein by reference in its entirety). Any covalently bound ligand or any ligand bound to a metal ion was removed because it cannot, at present, be modeled by the scoring function described herein. In addition, any “surface sugars” were removed as they are not typical of the problems encountered. This left a total of 103 cases (see Table 1 below). No further individual processing of the test cases was performed. (Note that the “Protein Data Bank” (PDB) is a database where target molecule structures are placed. The “PDB Code” is a four letter code that allows a given structure to be found and extracted from the PDB.)

TABLE 1

| | Number | Mini- | RMSD | | Number | Mini- | RMSD |
|---|---|---|---|---|---|---|---|
| PDB | of Rot | mum | of Top | PDB | of Rot | mum | of Top |
| Code | Bonds | RMSD | Score | Code | Bonds | RMSD | Score |
| 1aaq | 17 | 1.35 | 1.4 | 1lst | 5 | 0.58 | 1.43 |
| 1abe | 0 | 0.31 | 0.31 | 1mcr | 5 | 3.92 | 5.41 |
| 1acj | 0 | 0.59 | 0.71 | 1mdr | 2 | 0.41 | 0.78 |
| 1ack | 2 | 0.45 | 0.46 | 1mmq | 7 | 0.55 | 0.60 |
| 1acm | 6 | 0.31 | 0.31 | 1mrg | 0 | 0.45 | 3.42 |
| 1aha | 0 | 0.25 | 0.53 | 1mrk | 2 | 0.94 | 2.91 |
| 1apt | 18 | 1.10 | 1.63 | 1mup | 2 | 1.74 | 4.40 |
| 1atl | 9 | 1.05 | 4.24 | 1nco | 8 | 2.88 | 8.50 |
| 1azm | 1 | 1.40 | 2.33 | 1pbd | 1 | 0.29 | 0.38 |
| 1baf | 7 | 0.76 | 7.10 | 1poc | 23 | 2.81 | 8.62 |
| 1bbp | 11 | 1.45 | 1.55 | 1me | 21 | 8.83 | 10.14 |
| 1cbs | 5 | 0.70 | 12.63 | 1rob | 4 | 0.83 | 1.17 |
| 1cbx | 5 | 0.53 | 2.30 | 1snc | 5 | 1.17 | 5.60 |
| 1cil | 3 | 1.07 | 5.94 | 1srj | 3 | 0.48 | 0.58 |
| 1com | 3 | 0.76 | 0.76 | 1stp | 5 | 0.33 | 0.48 |
| 1coy | 0 | 0.52 | 0.70 | 1tdb | 4 | 1.33 | 7.09 |
| 1cps | 5 | 0.85 | 0.97 | 1tka | 8 | 1.44 | 1.44 |
| 1dbb | 1 | 0.72 | 0.85 | 1tng | 1 | 0.35 | 0.42 |
| 1dbj | 0 | 0.64 | 5.90 | 1tnl | 1 | 0.45 | 4.25 |
| 1did | 2 | 2.76 | 3.65 | 1tph | 3 | 0.63 | 1.44 |
| 1die | 1 | 2.24 | 2.30 | 1ukz | 4 | 0.43 | 6.20 |
| 1drl | 2 | 1.02 | 1.61 | 1ulb | 0 | 1.22 | 4.19 |
| 1dwd | 9 | 0.75 | 7.98 | 1wap | 3 | 0.29 | 0.34 |
| 1eap | 10 | 0.79 | 3.95 | 1xid | 2 | 0.79 | 4.23 |
| 1eed | 19 | 3.41 | 3.41 | 1xie | 1 | 0.34 | 3.89 |
| 1epb | 5 | 0.75 | 2.86 | 2ada | 2 | 0.53 | 0.58 |
| 1eta | 5 | 5.48 | 7.29 | 2ak3 | 4 | 1.91 | 3.24 |
| 1etr | 9 | 2.70 | 7.06 | 2cgr | 7 | 0.61 | 3.46 |
| 1fen | 4 | 0.98 | 2.45 | 2cht | 2 | 0.18 | 0.40 |
| 1fkg | 10 | 1.68 | 1.72 | 2cmd | 5 | 0.50 | 2.36 |
| 1fki | 0 | 0.30 | 0.54 | 2ctc | 3 | 0.36 | 4.15 |
| 1frp | 6 | 0.67 | 1.13 | 2dbl | 6 | 0.40 | 0.96 |
| 1ghb | 4 | 0.90 | 0.94 | 2gbp | 1 | 0.17 | 0.17 |
| 1glp | 10 | 1.45 | 8.92 | 2lgs | 4 | 0.71 | 5.48 |
| 1glq | 13 | 1.91 | 9.96 | 2phh | 1 | 0.51 | 0.51 |
| 1hdc | 6 | 1.52 | 11.25 | 2plv | 5 | 1.98 | 7.40 |
| 1hef | 19 | 3.63 | 5.29 | 2r07 | 15 | 1.17 | 2.45 |
| 1hfc | 10 | 1.37 | 7.77 | 2sim | 8 | 0.92 | 1.37 |
| 1hri | 9 | 1.49 | 3.29 | 2yhx | 3 | 1.07 | 6.99 |
| 1hsl | 3 | 0.76 | 2.21 | 3aah | 3 | 0.48 | 0.68 |
| 1hyt | 5 | 0.79 | 1.56 | 3cpa | 5 | 0.92 | 1.40 |
| 1icn | 15 | 1.78 | 9.43 | 3hvt | 1 | 0.27 | 0.56 |
| 1ida | 15 | 1.32 | 1.38 | 3ptb | 0 | 0.22 | 0.28 |
| 1igj | 3 | 0.90 | 7.46 | 3tpi | 6 | 0.42 | 0.53 |
| 1imb | 2 | 1.64 | 4.48 | 4cts | 3 | 0.73 | 0.77 |
| 1ive | 2 | 2.55 | 6.63 | 4dfr | 9 | 2.05 | 8.72 |
| 1lah | 4 | 0.71 | 0.77 | 4fab | 2 | 2.52 | 4.45 |
| 1lcp | 3 | 0.53 | 4.65 | 4phv | 12 | 0.38 | 0.38 |
| 1ldm | 1 | 0.80 | 5.24 | 6adp | 0 | 0.34 | 0.34 |
| 1lic | 15 | 1.32 | 4.39 | 7tim | 3 | 0.40 | 0.98 |
| 1lmo | 6 | 5.00 | 8.40 | 8gch | 7 | 1.70 | 4.45 |
| 1lna | 6 | 1.35 | 1.46 | | | | |

As expected, the rms deviation between the bound conformation (X=ray) and the closest computationally generated conformation increases with the number of rotatable bonds. In all but 5 cases, at least one conformation was generated by the conformational search with 1.5 Å rms deviation of the bound conformation. The most interesting aspect of the conformational search results is that for some of the more rigid ligands, the minimum rms deviation was large. For example, there are several ligands with fewer than five rotatable bonds, but with a minimum rms deviation near 1.0 Å. This occurs for two reasons. First, a clustering radius of 1.0 Å in all cases was used. This prevented the conformational space of small ligands from being sufficiently sampled. However,a clustering radius dependent on the molecule size could be used to alleviate this particular problem. The second problem is that a bond between two Sp² atoms was always treated as being conjugated. Thus, whenever this type of bond is encountered, it is strongly restrained to be planar. While bonds between two sp² atoms are often conjugated, this is clearly an over-simplification. This may be addressed, in accordance with the invention, by allowing the dihedral angles between two sp² atoms to deviate from planarity. This deviation can then be penalized according to the degree of conjugation. The penalty could be chosen crudely based on the types of the Sp² atoms (see, S. L. Mayo, B. D. Olafson, & W. A. Goddard, “DRIEDING: A Generic Force Field for Molecular Simulations”, J. Phys. Chem. 1990, Vol. 94, p. 8897).

For the docking runs, two different sets of parameters were tested to see their effects on the quality and speed of the docking runs: one for high quality docking and one for rapid searches. The key difference between the two sets of parameters are the match tolerance and the number and length of the BFGS optimization runs. The match tolerance ranges from 0.5 Å for the high quality to 0.25 Å for the rapid searches. Note that the larger the tolerance, the more matches will be found. Thus, a larger tolerance means a more thorough search, while a smaller tolerance denotes a less thorough but faster search. For the high quality runs, a maximum of 100 matches per ligand were optimized for 100 steps compared to 25 matches per ligand for 20 steps for the rapid searches.

The first problem is to generate at least one docked position between a given rms deviation cutoff. Here, terminology is adopted that a ligand that is docked to within x Å of the crystallographically observed position of the ligand is referred to as an x Å hit. The rms deviations are shown for the high quality runs in Table 1. For the high quality runs, 89 of the 103 cases produce at least one 2.0 Å hit. The numbers drop to 80 at 1.5 Å, 63 at 1.0 Å and 26 at 0.5 Å. For the rapid searches, 75 of the 103 cases produce a 2.0 Å hit, 65 produce a 1.5 Å hit, 42 produce a 1.0 Å hit and 16 produce a 0.5 Å hit. In both cases, these numbers compare favorably with similar statistics from other docking packages that have been tested on the Gold or similar test sets (see, Jones, G., et al., Development and Validation of a Generic Algorithm for Flexible Docking,“Journal of Molecular Biology, 1997, Vol. 267, p. 727-748; Baxter, C. A. et al., “Flexible Docking Using Tabu Search and an Empirical Estimate of Binding Affinity,” PROTEINS: Structure, Function, and Genetics, 1998, Vol. 1998, p. 367-382; Rarey, M., B. Kramer, and T. Lengauer, “The Particle Concept: Placing Discrete Water Molecules During Protein-ligand Docking Predictions,” PROTEINS: Structure, Function, and Genetics, 1999, Vol. 34, p. 17-28; Rarey M., B. Kramer, and T. Lengauer, “Docking of Hydrophobic Ligands With Interaction-based Matching Algorithms,” Bioinformatics, 1999, Vol. 15(3), p. 243-250; and Kramer, B., M. Rarey, and T. Lengaeur, “Evaluation of the FlexX Incremental Construction Algorithm for Protein-Ligand Docking,” PROTEINS: Structure, Function, and Genetics, 1999, Vol. 37, p. 228-241).

The second problem is to correctly rank the docked compounds; i.e., is the top ranked conformation reasonably close to the crystallographically observed position for the ligand? This is a significantly more difficult problem than the first. The rms deviation between the top scoring docked position and the observed position for the high quality runs are given in Table 1. In this case, there is little difference between the two sets of parameters. For the high quality runs, 48 of the 103 cases produce a 2.0 Å hit as the top scoring docked position. This number drops to 41 at 1.5 Å, 34 at 1.0 Å and 10 at 0.5 Å. For the rapid searches, 45 of the 103 cases produce a 2.0 Å hit as the top scoring docked position with 41 at 1.5 Å, 34 at 1.0 Å and 10 at 0.5 Å.

The utility of the scoring function used in this study lies less as a tool to absolutely rank the docked conformations than as an initial filter to select only a few docked conformations. Most of the well docked positions, i.e., low rms deviations, survive this 10% cutoff. Most of the docked positions, however, do not. For the high quality runs, on average 74 positions are found, but after the 10% cutoff on average only 8 remain. For the rapid searches, on average nearly 21 positions are found, but after the cutoff on average only 5 remain. At this point, the docked positions that survive the 10% score cutoff could be further optimized, visually screened, or passed to a more accurate, but less efficient scoring function.

For the high quality runs, the average CPU time (e.g., using a Silicon Graphics Incorporated (SGI) computer R12000) per test case is approximately 4.5 seconds. At this rate, screening one million compounds with one CPU would take about 50 days. For the rapid searches, the average CPU time per test case drops to approximately 1.1 seconds per test case. At this rate, screening one million compounds with one CPU would take about 12 days. Because database docking is a highly parallel job, multiple CPUs could easily cut this to a reasonable amount of time (for example, a day or so).

In this section, a few of the successful cases are shown to demonstrate the strengths of the approach described herein to docking small molecules. In all of these cases, the results shown are from the medium quality docking runs. The first case is the dipeptide Ile-Val from the PDB entry 3tpi (see, Marquart, M., et al., “The Geometry of the Reactive Site and of the Peptide Groups in Trypsin, Trypsinogen and Its Complexes With Inhibitors,” Acta Crystallographica, 1983, Vol. B39, p. 480, which is hereby incorporated herein by reference in its entirety). This case has no clear anchor fragment and as a result, the incremental construction approach to docking might have difficulties with this ligand. Our conformational search procedure produced a conformation within 0.42 Å of the observed conformation. The rms deviation between the best scoring docked position and the observed position is 0.53 Å.

The second example, with a ligand having 15 rotatable bonds, is a much more difficult example. It is an HIV protease inhibitor from the PDB entry lida (see, Tong, L., et al., “Crystal Structures of HIV-2 Protease In Complex With Inhibitors Containing Hydroxyethylamine Dipeptide Isostere,” Structure, 1995, Vol. 3(1), p. 33-40, which is hereby incorporated herein by reference in its entirety). In this case the conformational search procedure was able to generate a conformation with an rms deviation of 0.96 Å from the bound conformation. The rms deviation for the top scoring docked position is 1.38 Å. In fact, the top 13 scoring docked positions are all within 2.0 Å of the observed position with the closest near 1.32 Å.

The final case is an HIV protease inhibitor from the PDB entry 4phv (see, Bone, R., et al., “X-ray Crystal Structure of The HIV Protease Complex With L-700, 417, An Inhibitor With Pseudo C2 Symmetry,” Journal of the American Chemical Society, 1991, Vol. 113 (24), p. 9382-9384, which is hereby incorporated herein by reference in it entirety). The ligand in this case has 12 rotatable bonds. This clearly demonstrates the value of including the final flexible gradient optimization step of the ligand. The closest conformation produced from the conformational search procedure is 1.32 Å from the crystallographically observed conformation. With an rms deviation of 0.38 Å, the top scoring docked position is also the closest to the observed position. The smallest rms deviation that could have been obtained without the flexible optimization is that of the closest conformation generated by the conformational search procedure, i.e., 1.32 Å. Thus, in this case, the flexible optimization decreased the final rms deviation by at least 1.0 Å.

It is often assumed that when docking simulation fails, the score has failed, i.e., the global minimum of the scoring function did not correspond to the crystallographically determined position for the ligand. Since the docking problem involves many degrees of freedom, it is reasonable to believe that in many cases the failure can be attributed to insufficient search. It is the goal of this section to identify the cause of failure in the cases in which the procedure described herein performed poorly.

To classify docking failures as either scoring failures or search failures, the ligand was taken as bound to the target molecule and a BFGS optimization was performed. If the resulting score was significantly less than the best score found from the docking runs, the failure is classified as a search failure. Every other failure is classified as a scoring failure.

The vast majority of the cases qualify as moderate scoring errors, i.e., the global minimum appears not to correspond to the crystallographic position of the ligand, but the percent difference between the global minimum and the best score near the crystallographic position of the ligand is less than 10%. In these cases, it is difficult to decide which aspects of the score are failing, but it is reasonable to believe that many of these cases can be corrected simply by including some more detail in the scoring function, such as angular constraints on the hydrogen bonding term or a salvation model. There are, however, a few cases with dramatic scoring errors. These cases provide some insight into the weakness of the score and the complexities of target molecule/ligand interactions.

The case 1glq (see, Garcia-Saez, I., et al., “Molecular Structure at 1.8 A of Mouse Liver Class pi Glutathione S-Transferase Complexed With S-(p-Nitrobenzyl)Glutathione and Other Inhibitors,” Journal of Molecular Biology, 1994, Vol. 237, p. 298-314) pointed out the main weakness of the score used in this study—hydrogen bonding patterns. This is a polar ligand. The top ranked position for this ligand scores very well largely because there are many “perceived” hydrogen bonds. In reality, these hydrogen bonds would be extremely weak because the angular dependence of the interaction is poor. Moreover, the sulfur atom in the X-ray position is accepting a hydrogen bond from the OH of a tyrosine and the carboxylic acid is involved in a salt bridge with a lysine. Neither of these interactions was recognized by the scoring function described herein.

In the case live (see, Jedrzejas, M. J., et al., “Structures of Aromatic Inhibitors of Influenza Virus Neuraminidase,” Biochemistry, 1995, Vol. 34, p. 3144-3151), the correct position receives a relatively poor score largely due to the estimated strain of the observed conformation. The docking procedure recognizes certain bonds as being conjugated. Thus, a stiff penalty is applied when these bonds are not planar. In the observed conformation, the dihedral angles are all nearly 80° from planar. If these dihedral angles are forced to be near 0°, the conformation is no longer compatible with the observed interactions between the ligand and the target molecule. It would be difficult for any docking algorithm to predict these values for the dihedral angles.

The case 1hef (see, Murthy, K. H. M., et al., The Crystal Structures at 2.2-A Resolution of Hydroxyethylene-Based Inhibitors Bound to Human Immunodeficiency Virus Type 1 Protease Show That The Inhibitors are Present in Two Distinct Orientations,”Journal of Biological Chemistry, 1992, Vol. 267, p. 22770-22778), an HIV protease inhibitor, is perhaps the most interesting of all of the dramatic scoring errors. The binding pocket is at the interface of a dimer with the target monomers being related through a crystallographic symmetry operation. At the C-terminus of the ligand, a methyl group is within 2.0 Å. These interactions would be extremely difficult to predict. Our program did come up with an interesting alternate conformation for the C-terminus of the ligand. This conformation eliminates both the internal and external steric clashes and forms an additional hydrogen bond with the target molecule.

There are two cases that can be classified as conformational search failures: 1hef and 1poc. In these cases the best conformation produced is 2.1 Å and 2.3 Å, respectively. The ligand in the case 1poc has 23 rotatable bonds, and thus, it is very difficult to fully cover its conformational space with only 50 conformers. While the ligand in the case 1hef is also very flexible (18 rotatable bonds), the observed conformation, as described above, also has a serious steric clash. Thus, this is, as should be expected, a very difficult challenge for any conformational search procedure.

In this application, a new rapid technique for docking flexible ligands into the binding sites of target molecules is presented. The method is based on a pre-generated set of conformations for the ligand and a final flexible gradient based optimization of the ligand in the binding site of the target molecule. Based on the results, this is a robust approach to handling ligand flexibility. With relatively few conformations (less than 50 per molecule), usually a conformation within 1.5 Å of the bound conformation can be generated. Applying the flexible optimization as the final step reduces the number of conformations required while maintaining high quality final docked positions.

There are opportunities to improve the exemplified docking technique. Such improvements also fall within the scope of the present invention. For example, the conformer generation, while reasonably successful, should treat small relatively rigid molecules and large flexible molecules differently. Since the conformational space of very large flexible molecules is too large to explore thoroughly, a Monte Carlo search algorithm is used. In addition, the score used to rank the conformations is certainly simplistic and can be improved. For example, variations of solvation models (see, Eisenberg, D. and A.D. McLachlan, “Solvation Energy in Protein Folding and Binding,” Nature, 1986, Vol. 319, p. 199-203; Still, W. C., et al., “Semianalytical Treatment of Solvation For Molecular Mechanics and Dynamics,” Journal of the American Chemical Society, 1990, Vol. 112, p. 6127-6129, both of which are hereby incorporated herein by reference in their entirety) would likely give better conformations. Finally, a better treatment of strain, particularly that for rotation about bonds between two Sp² atoms, might lead to improved results.

In the embodiment exemplified, the algorithm used to find the polar hot spots tends to find any hydrogen bond donor and acceptor rather than those buried in the binding site. Improving the hot spot search routine will not only increase the quality of the technique, but will also decrease the number of hot spots needed and, thus, make the technique more efficient. Some available programs, such as GRID (see, Goodford, P. J., “A Computational Procedure for Determining Energetically Favorable Binding Sites on Biologically Important Macromolecules,” Journal of Medicinal Chemistry, 1985, Vol. 28(7), p. 849-857; and Still, W. C., et al., “Semianalytical Treatment of Solvation For Molecular Mechanics and Dynamics,” Journal of the American Chemical Society, 1990, Vol. 112, p. 6127-6129, both of which are hereby incorporated herein by reference in their entirety) or the LUDI binding site description (see, Bohm, H. J., “LUDI: Rule-based Automatic Design of New Substituents For Enzyme Inhibitor Leads,” Journal of Computer-Aided Molecular Design, 1992,“Vol. 6, p. 693-606, which is hereby incorporated herein by reference in its entirety) or a documented method (see, Mills, J. E. J., T. D. J. Perkins, and P. M. Dean, “An Automated Method For Predicting The Positions of Hydrogen-bonding Atoms In Binding Sites,” Journal of Computer-Aided Molecular Designs, 1997, Vol. 11, p. 229-242, which is hereby incorporated herein by reference in its entirety) would likely show some improvement. In addition, separating the polar hot spots into donor, acceptor, ionic, etc., hot spots might improve the results. Finally, in a practical application, most users would be willing to spend some time to enhance the image, i.e., eliminate by hand bad hot spots, and add hot spots where needed. In practice, this will significantly improve docking runs.

In all docking programs, a good score should be efficient, error tolerant, and accurate. The score used here satisfies the first two qualities. These two qualities, however, are usually not compatible with the third. It appears that this score will still be useful as an initial screen after which a more accurate score can be applied. Geometric constraints for the hydrogen bonding term, recognition of ionic interactions and solvation effects, and terms for dealing with metals can be introduced to improve accuracy.

Nonetheless, when a crystal structure is available, the approach of the present invention to molecular docking is useful in library screening prioritization. Even with lower quality structural information, such as a homology model, the technique described herein will still provide useful information.

After each ligand is docked to the target, docking results may be organized using a clustering procedure to facilitate analysis. In this procedure, multiple clusters are formed, each of which is made up of a group of similar positions of the ligand, with respect to the target molecule. A single linkage clustering algorithm may be used, with the rms deviation between pairs of ligand positions as the clustering metric. Pairs of positions wherein the rms deviation between the cores of the ligands are less than some predetermined number, typically 0.25 Å to 0.5 Å, are in the same cluster. Alternative clustering algorithms may also be used; single linkage clustering may be advantageous in a particular case because of its simplicity. The relative number of compounds in the library in the top cluster is a measure of the library's complementarity to the target molecule, and is used to rank the library.

In one embodiment, the ligand positions are clustered using a graphical method. For a library with N compounds, the clustering procedure requires N(N-1)/2 rms deviation calculations. For a ten-thousand member library with one pose per compound, fifty thousand rms deviation calculations are required. This number can be drastically reduced in practice by the following considerations. If the distance between the center of mass of the cores of two poses is greater than a predetermined cutoff, then the rms deviation between the two cores will necessarily be greater than the rms deviation cutoff. Therefore, a grid defining a three-dimensional volume sub-divided into smaller volume units is placed around the binding site of the target molecule. The center of mass of each of the poses is calculated, and is associated with a particular grid cube. Rms deviations are calculated only between positions in nearby cubes. In practice, this decreases the number of calculations by a factor of 10-100.

One potential difficulty with using a docking approach to address the library prioritization problem is false positives. This problem is best illustrated through an example. Suppose we have two combinatorial libraries (A and B) each of which contains 10000 compounds. Suppose now that against some target library A contains no active compounds whereas library B contains 25 active compounds. Finally, assume that we have a docking procedure that is sufficiently accurate to classify compounds correctly (active or inactive) 95% of the time. Then, from library A, we would on average find 500±22 hits whereas for library B we would on average find 524±22 hits. Thus, even with this extremely accurate docking procedure, there would still be a significant chance of classifying library A as more active than library B. In addition, no docking method is 95% accurate. Also, there is significant structural similarity between the compounds in a combinatorial library, and so, a library with active compounds will contain a significant number of compounds similar to the active compounds of the library. These compounds similar to active compounds will be more likely found as false positives by any computational procedure.

This effect again is best illustrated with an example. Suppose that the binding site of the target has three pockets P1, P2, and P3 and that the core of the library has three positions for substitutions R1, R2, and R3 (see FIG. 9). Suppose further that at each position there are 30 different synthons for a total of 27000 compounds. Finally assume that a compound from this library is active if and only if it has one of three synthons at R1, one of three synthons at R2 and one of three synthons at R3 giving this library 27 active compounds. Even if these 27 active compounds are well docked and receive good scores it is unlikely that the scores of these 27 active compounds would cause this library to stand out from inactive libraries.

However, there are 756 compounds that have at least two of the “active” synthons. These compounds are much more likely to receive a better than random score. Thus, even with a less accurate docking procedure, it is likely that regions of chemistry space, as represented by combinatorial libraries, can be identified correctly.

### EXAMPLES

The clustering method of the present invention was evaluated in comparison to a scoring method using four ECLiPS™ aspartyl protease inhibitor libraries, PL 419, PL 444, PL 792, and PL 799, available from Pharmacopeia, Inc. These libraries were docked into the binding sites of plasmepsin II (pdb identifier 1sme) and cathepsin D (pdb identifier 1lyb). The four libraries are based on the core of Pepstatin, shown below:

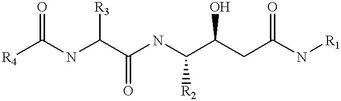

### Pepstatin Common Core

### ECLiPS™ Aspartyl Protease Inhibitor Libraries

These libraries were chosen because three of the four (PL 444, PL 792, and PL 799) have previously been screened for activity against both plasmespin II and cathepsin D and produced a significant number of active compounds. The fourth library, PL 419, has been tested against plasmespin II, yielding a significant number of active compounds, and although it has not been tested against cathepsin D, a compound re-synthesized from the library was active against cathepsin. In addition, as the libraries consist of large (mean molecular weight of 550) and flexible (mean rotatable bond count of 19) compounds, they represented a significant challenge to any docking procedure. Relevant physical properties of the libraries are shown in Table 3, including molecular weight, number of rotatable bonds, and number of compounds in the library.

Data from the high throughput screens of the libraries against the two targets, and from determination of the Kᵢ's of re-synthesized compounds from the libraries are shown in Table 4. The libraries may be ranked as to relative activity according to these data.

Data from high throughput screens generally takes the form of active and inactive, that is whether a given compound is found on a “decoded” synthesis bead showing positive activity in the screening test. Because a single decoded bead has a fair chance of being a false positive, it can be difficult to assign an absolute degree of activity/potency to a library based on high throughput data. Compounds that appear on multiple decoded beads, or “duplicate decodes”, are much less likely to be false positive. (The number of beads screened is usually greater than the number of compounds, typically by a factor of three, in order to minimize noise). Thus, the number of duplicate decodes is a better indication of the activity of a library.

TABLE 3
Library Physical Property Distributions

| | Molecular | Rotatable | |
|---|---|---|---|
| | Weight | Bonds | Number of |
| Library | (std dev) | (std dev) | Compounds |
| PL419 | 521 (81) | 16.3 (3.3) | 5580 |
| PL444 | 584 (68) | 19 (2.5) | 25200 |
| PL792 | 530 (75) | 18.9 (2.9) | 13020 |
| PL799 | 662 (91) | 20.4 (3.0) | 18900 |

A second measure of the activity/potency of a library is the potency of those decoded compounds that are re-synthesized and assayed. In most cases, only a handful of the decoded compounds have been re-synthesized in larger amounts and assayed. Thus, the potency of the re-synthesized compound by itself, is not a perfect reflection of the overall activity of the library. Thus the activity of a library is measured by both the number of decodes/number of duplicate decodes and the potency, typically maximum potency of selected re-synthesized compounds.

With regard to their activity/potency towards plasmepsin, the libraries are ranked as follows:

PL 792>PL 419=PL 444>PL 799

Relative activity/potency is defined in this manner based on the number of decodes/number of duplicate decodes and the value of Kᵢ shown in Table 4. PL 419 and PL 792 both produced a significant number of decodes and duplicate decodes. PL 792 produced several compounds with Kᵢ(s) at or below 100 nM whereas the most potent compound found in PL 419 had a Kᵢ of 540 nM. Thus, PL 792 is ranked as the most active library. Because it produced more decodes and duplicate decodes, PL 419 is ranked as more active against plasmepsin than PL 799. PL 444 produced a similar number duplicate decodes as PL 799, but produced a significantly more potent compound. Thus, PL 444 was rated as more active than PL 799. PL 444 and PL 419 are ranked as roughly equally active because PL 419 produced significantly more duplicate decodes, but PL 444 produced a significantly more potent compound.

For cathepsin, the libraries were ranked as:

PL 444>PL 792>PL 799

PL 444 produced the most duplicate decodes and the most active compound, and is therefore rated as the most active against cathepsin. PL 792 produced more duplicate decodes and more potent compounds than did PL 799. Thus, against cathepsin PL 792 is rated as more active than PL 799. PL 419 was not screened against cathepsin, but it produced a compound which was significantly more potent against Cathepsin than any produced by PL 799.

TABLE 4
Library Activity and Potency

| | Plasmepsin | | Cathepsin | | |
|---|---|---|---|---|---|
| | Number of | | Number of | | |
| | Decodes/ | | Decodes/ | | |
| | Number of | | Number of | | |
| | Duplicate | Maximum | Duplicate | Maximum | |
| Library | Decodes^{a} | Potency^{b} | Decodes^{a} | Potency^{b} | |
| PL419 | 106/68 | 540 nm | — | 230 | nm |
| PL444 | 61/17 | 140 nm | 78/36 | 21 | nm |
| PL792 | 134/57 | 50 nm | 93/20 | 50 | nm |
| PL799 | 36/15 | 490 nm | 50/13 | 1100 | nm |

a) The number of decodes is followed by the number of duplicate decodes.
b) The maximum observed potency of all resynthesized compounds.

In addition, eight “virtual” libraries were created as negative controls, differing from the positive controls only in the configuration of a single chiral center in the statine core. These virtual libraries were designated PL 419R, PL 419D, PL 444R, PL 444D, PL 792R, PL 792D, PL 799R and PL 799D. The original pepstatin scaffold, shown above, corresponds to the statin core, and has two stereocenters, the hydroxyl bearing carbon and the Cα atom of the amino acid. Both stereocenters are in the L configuration. The libraries designated with an appended R are identical to the standard libraries, except that the carbon bearing the hydroxyl group has a configuration opposite to that in the positive control, designated as R, shown below:

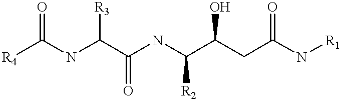

### D-amino Acid Common Core

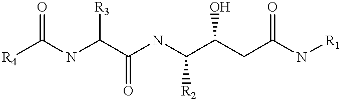

### R-statine Common Core

The libraries designated with an appended D are identical to the standard libraries, except that the statine piece has a D-amino acid instead of the standard L-amino acid, shown above. These virtual libraries are utilized as negative controls because there are no R-statine or D-amino compounds known to exhibit activity against plasmepsin II or cathepsin D. Therefore, it was assumed that these additional libraries either would be significantly less active than the original libraries or completely inactive. In addition, because these libraries have exactly the same property distributions (molecular weight, number of rotatable bonds, hydrogen bond donors, etc.), differences between the results of docking the negative control libraries and the original libraries are directly attributable to differences in fit and complementarity with the receptor.

Each of the twelve libraries was docked into the binding site of plasmepsin 2 and cathepsin D using the procedure described in U.S. application Ser. No. 09/595,096. In the case of plasmepsin, a box 20 Å×32 Å×22 Å around the binding site was chosen as the search space. For cathepsin D, a box 22 Å×30 Å×24 Å around the binding site was chosen as the search space. For simplicity, only the top ranked docked pose for each molecule was used in the analysis. The docking times for both cases range from 3-5 seconds per compound (see Table 5). Results were analyzed by both a scoring method (comparative) and by the clustering method of the present invention.

TABLE 5
Docking Times for Plasmepsin and Cathepsin
Libraries

| Target | | | | Docking Times (sec) |
|---|---|---|---|---|
| | Plasmepsin | 419 | Orig. | 4.1 |
| | | | R | 4.1 |
| | | | D | 4.0 |
| | | 444 | Orig. | 5.7 |
| | | | R | 4.7 |
| | | | D | 4.7 |
| | | 792 | Orig. | 4.9 |
| | | | R | 4.8 |
| | | | D | 4.5 |
| | | 799 | Orig. | 5.1 |
| | | | R | 3.4 |
| | | | D | 3.8 |
| | Cathepsin | 419 | Orig. | 3.4 |
| | | | R | 3.4 |
| | | | D | 3.4 |
| | | 444 | Orig. | 4.9 |
| | | | R | 3.9 |
| | | | D | 3.9 |
| | | 792 | Orig. | 4.2 |
| | | | R | 4.1 |
| | | | D | 3.9 |
| | | 799 | Orig. | 4.5 |
| | | | R | 3.1 |
| | | | D | 3.2 |

### Example 1 (Comparative): Scoring Analysis

The scoring method compares score distributions between libraries. The root mean square (rms) of the scores in the top 5% of the docked compounds (as ranked by score) is used as an overall library score. The rationale is that if a library has active compounds, then a significant number of the compounds should be sufficiently similar to the active compounds that they should fit reasonably well into the binding site and receive similarly good scores. Thus, the top scoring compounds from an active library should be distributed differently than those from an inactive library.

To analyze the results using the score, first the compounds are sorted according to their score. A library score is then calculated via

$$Library Score = {( \frac{20}{N} {\sum }_{i} {S}_{i}^{2} )}^{1 / 2} \qquad ( 1 )$$

where Sᵢ is the score of the ith ranked compound, the sum extends only over the top 5% of compounds, and N is the number of compounds in the library. The factor of 20 appears in equation (1) because the sum is over only one twentieth (5%) of the compounds in the library. The scoring procedure described above was used. The reason for choosing the root mean square (rms) of the scores rather than the mean is that the rms will favor those libraries with a few compounds that receive very good scores.

There are a number of additional statistical quantities that could be used to analyze the scores. For example, Goddon et al., Statistical Analysis of Computational Docking of Large Compound Data Bases to Distinct Protein Binding Sites, the skewness of the score distribution from a large number of docked compounds was examined over a range of targets. Additional statistical measures including the mean and standard deviation of all the scores could be used. The problem with using statistical quantities, such as the mean, standard deviation, or skewness, is that they are all affected by the compounds that receive poor scores whereas we are interested in the compounds that receive good scores. For example, a library whose compounds all receive mediocre scores will have the same mean as a library half of whose compounds receive low scores and half receive high scores. We would be much more interested in the second library. Since we are primarily concerned with the compounds that receive good scores, only the top 5% of the compounds are used. The exact choice of 5% was arbitrary but seemed to have little bearing on the conclusions.

For the docking of PL419, PL444 and PL792 into plasmepsin and cathepsin, the score ranks the original libraries as the best followed by the library with the R-statine core, and then by the library with the D-amino acid (see Table 6). For PL799 with both plasmepsin and cathepsin, the score again ranks the original library as the best but of the three but it ranks the library with the D-amino acid second and the library with the R-statine core last. Thus as was expected for both targets and all three libraries, the library that scores the best, as judged by equation 1, is the original library.

TABLE 6
Score and Cluster Rank

| | | | | Largest |
|---|---|---|---|---|
| Library | | | Library Score | Cluster |
| Plasmepsin | PL419 | Orig. | 162.5 | 0.23 |
| | | R | 159.3 | 0.16 |
| | | D | 158.7 | 0.07 |
| | 444 | Orig. | 173.5 | 0.34 |
| | | R | 171.0 | 0.25 |
| | | D | 167.7 | 0.12 |
| | 792 | Orig. | 172.1 | 0.34 |
| | | R | 169.7 | 0.16 |
| | | D | 161.8 | 0.03 |
| | 799 | Orig. | 167.0 | 0.12 |
| | | R | 165.0 | 0.06 |
| | | D | 165.3 | 0.03 |
| Cathepsin | 419 | Orig. | 170.0 | 0.33 |
| | | R | 162.3 | 0.08 |
| | | D | 160.9 | 0.02 |
| | 444 | Orig. | 178.9 | 0.45 |
| | | R | 175.0 | 0.19 |
| | | D | 168.9 | 0.07 |
| | 792 | Orig. | 175.3 | 0.36 |
| | | R | 174.2 | 0.18 |
| | | D | 168.7 | 0.13 |
| | 799 | Orig. | 170.1 | 0.08 |
| | | R | 167.0 | 0.02 |
| | | D | 168.0 | 0.03 |

The comparison across the four original libraries is less straightforward. For example, the score for a compound being docked often shows some correlation with the physical properties, such as molecular weight, number of polar atoms etc., of the compound. In particular, bigger and more polar molecules tend to get better scores simply because they have more atoms making stronger interactions. For plasmepsin, the score ranks PL444 as clearly the best followed by PL792, then by PL799 and finally by PL419. For cathepsin, the score again ranks PL444 as the best followed by PL792 and then by PL419 and PL799. Thus, there appears to be some correlation between the degree of actual activity of a library (see Table 4, above) and the score (Table 6). One should note, however, that for plasmepsin the versions of PL444 and PL792 with the R-statine (PL444R and PL792R) were ranked higher than the original version of PL419. Thus, the correlation is not perfect.

The score can also be used to rank individual synthons. To assign a score to a given synthon, equation (1) is applied only to those compounds containing the given synthon. For this we restrict our attention to PL792 and plasmepsin. For the R₂ substitution there are three synthons: (1) —Ch₂Ph, (2) —CH₃, and (3) —CH₂CH(CH₃)₂. A significant number of actives where found with both synthons (1) and (3) but none were found with (2). The scores for these synthons are 169.9, 155.2, and 170.6, respectively. Based on the SAR this is the correct ranking: synthon (1) and (3) are closely ranked with synthon (2) being ranked significantly lower.

The agreement with the SAR and the score for the R3 synthons is not nearly as perfect. In fact there appears to be no correlation. Most of the top ranked synthons are the large and polar amino acids whereas the small apolar amino acids dominate the SAR. There are a couple explanations for this lack of correlation. First, there is a noted correlation between the size and polarity of the molecule and the score. If we restrict our attention to the small apolar amino acids then the score ranks them L-leucine>L-isoleucine>L-valine>L-alanine>L-t-butylglycine>D-leucine>D-alanine, where L-valine and L-isoleucine are the most commonly observed synthons among the experimentally observed active compounds. Thus, within the set of apolar amino acids some correlation with the experimental SAR is observed. A second reason for the lack of correlation is that, since there are 31 R3 synthons there are only 420 molecules containing each synthon. As a result, the score for each synthon is based on only 21 compounds (top 5% of compounds). This likely introduces a significant amount of noise, which reduces the ability to score the various R3 synthons accurately.

With the scoring method it is difficult to compare libraries with very different physical properties because the score is correlated with properties such as molecular weight and polarity. This problem was best illustrated through the analysis of the R3 synthons of PL792. In this case the SAR clearly showed that small L-amino acids are preferred at this position. The best scoring synthons, however, were generally the large polar amino acids. When restricted to small hydrophobic amino acids the score showed some correlation with the high throughput SAR for the R3 synthons. This problem might be mitigated through the use of an accurate solvation model, though to be of use the model would also have to be fast and error tolerant.

### Example 2: Clustering Analysis

For the clustering analysis, the clusters were formed using single linkage clustering where the rms deviation between the cores of two docked molecules was used as the metric. Essentially, any two poses whose cores are within some pre-determined cutoff, typically 0.25 Å to 0.5 Å, are in the same cluster. For this study a 0.5 Å cutoff was used. The percentage of compounds from the library in the top cluster was used to rank the library. Single linkage clustering was used to facilitate the computations requiring no parameters other than the rms deviation cutoff. This was sufficient to demonstrate the utility of clustering to extract information from the results of docking large combinatorial libraries.

As a measure of the quality of fit, the percentage of the compounds in the largest cluster was used. As with the score ranking, the original library for both targets and all three libraries were ranked higher than the corresponding R-statine or D-amino acid versions (see Table 4). The clustering appears to better separate the original libraries from the control libraries than did the score. The closest cluster size between an original library and one of the control libraries is with PL419 and PL444 and plasmepsin. For these two cases, the top cluster for the original library is only 30-40% larger than the top cluster for the corresponding R-statine version of the library. In the remaining six cases the top cluster size with the original library is at least double that of the control libraries.

As with the score ranking, the cluster ranking over the different libraries is more problematic. For both plasmepsin and cathepsin, the cluster size correctly ranks PL792 as the best of the three, followed by PL419 and then by PL799. The cluster size, however, incorrectly ranks the R-statine versions of PL419, PL444 and PL792 ahead of the original version of PL799. This can be attributed to the differences in physical properties between the libraries. The compounds in PL799 are significantly bigger and more flexible (see Table 3) than those in PL419 and PL792. In addition, there is a central flexible ring in the compounds in PL799²⁹ which makes the conformational analysis more difficult. Thus the compounds in PL799 are much more difficult to dock correctly, leading to a lower percentage of correctly docked compounds and as a result a smaller top cluster.

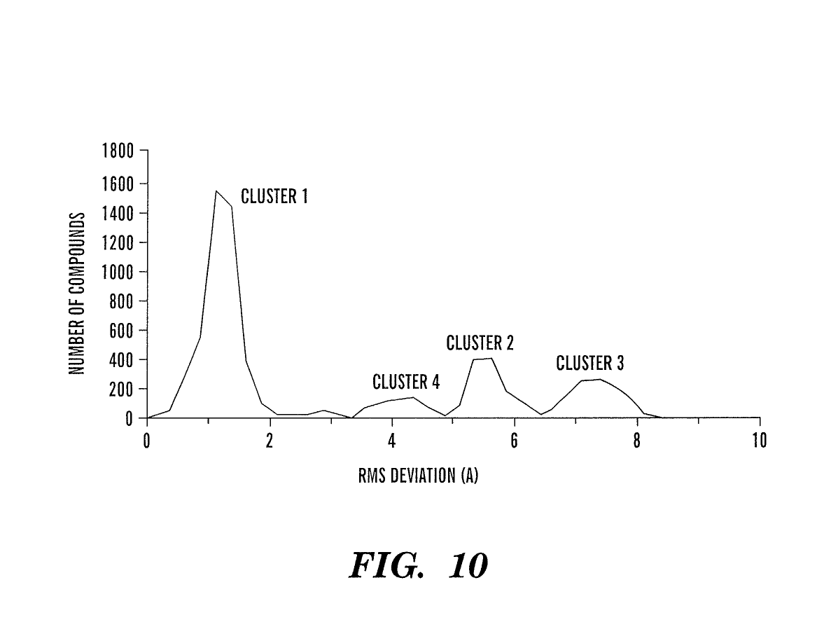

The clustering method is also extremely useful as a data reduction technique. Both the plasmepsin crystal structure, 1sme, and the cathepsin crystal structure, 1lyb, used in this study contain pepstatin in the binding site. As mentioned above, the core of each of these libraries was based on the core of pepstatin. As a result, a direct rms deviation can be calculated between the core of each of the docked compounds and the crystallographically observed binding mode of the core of pepstatin. For PL792 and plasmepsin, a graph of the number of compounds having a particular rms deviation is shown for each cluster of significant size (more than 100 members) in FIG. 10. This shows that there are relatively few significant clusters and that the top cluster is correctly docked. The same can be said for all four of the original libraries for both targets: the top cluster is docked correctly, and there are relatively few significant clusters (see Table 7). In cases where visual screening is used to further filter the docked compounds, clustering can reduce the effort required from examining tens of thousands of individual compounds to examining several clusters.

The clustering method is more advantageous than the scoring method because it relies less on the accuracy of the score. Rather, it depends on the ability to accurately and consistently dock compounds, and it is generally easier to correctly dock compounds than to accurately predict binding affinities.

TABLE 7
Cluster Sizes.^{a,b}

| | | | Cluster | Cluster | Cluster | Cluster |
|---|---|---|---|---|---|---|
| Target | Library | | 1 | 2 | 3 | 4 |
| Plasmepsin | 419 | Orig. | 0.220 | 0.075 | 0.065 | 0.035 |
| | | R | 0.178 | 0.089 | 0.051 | 0.037 |
| | | D | 0.077 | 0.047 | — | — |
| | 444 | Orig. | 0.336 | 0.121 | 0.076 | 0.0275 |
| | | R | 0.245 | 0.135 | 0.043 | 0.029 |
| | | D | 0.123 | 0.064 | 0.050 | 0.029 |
| | 792 | Orig. | 0.340 | 0.094 | 0.082 | 0.039 |
| | | R | 0.164 | 0.106 | 0.088 | 0.032 |
| | | D | 0.034 | 0.028 | 0.028 | 0.024 |
| | 799 | Orig. | 0.118 | 0.062 | 0.028 | 0.013 |
| | | R | 0.057 | 0.024 | 0.020 | 0.006 |
| | | D | 0.035 | 0.026 | 0.014 | 0.007 |
| Cathepsin | 419 | Orig. | 0.345 | 0.032 | 0.020 | — |
| | | R | 0.090 | 0.031 | 0.026 | 0.024 |
| | | D | 0.020 | — | — | — |
| | 444 | Orig. | 0.456 | 0.070 | 0.063 | 0.031 |
| | | R | 0.192 | 0.156 | 0.070 | 0.033 |
| | | D | 0.070 | 0.053 | 0.045 | 0.037 |
| | 792 | Orig. | 0.363 | 0.151 | 0.081 | 0.034 |
| | | R | 0.188 | 0.124 | 0.064 | 0.056 |
| | | D | 0.133 | 0.047 | 0.037 | 0.035 |
| | 799 | Orig. | 0.076 | 0.055 | 0.033 | 0.022 |
| | | R | 0.015 | 0.015 | 0.013 | 0.010 |
| | | D | 0.034 | 0.011 | 0.007 | 0.0065 |

¹⁾The clusters are given as fractions of the entire library.
²⁾The cluster in bold is the correct cluster as judged by the rms deviations (<2.0Å) between the members of the cluster and the crystallographically observed position for pepstatin.

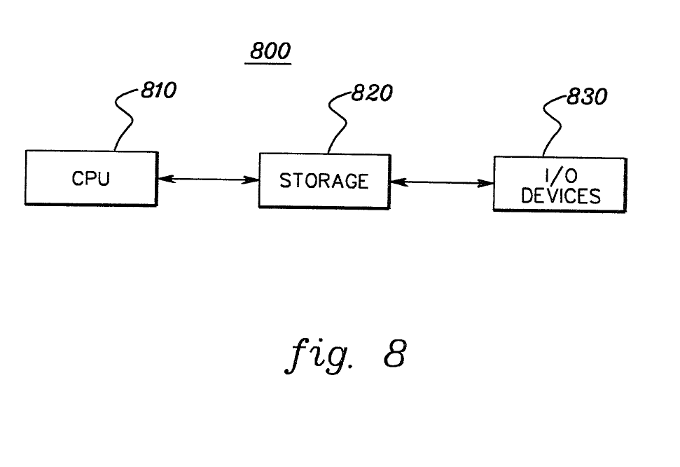

The capability of the present invention can readily be automated by creating a suitable program, in software, hardware, microcode, firmware or any combination thereof. Further, any type of computer or computer environment can be employed to provide, incorporate and/or use the capability of the present invention. One such environment is depicted in FIG. 8 and described in detail below.

In one embodiment, a computer environment 800 includes, for instance, at least one central processing unit 810, a main storage 820, and one or more input/output devices 830, each of which is described below.

As is known, central processing unit 810 is the controlling center of computer environment 800 and provides the sequencing and processing facilities for instruction execution, interruption action, timing functions, initial program loading and other machine related functions. The central processing unit executes at least one operating system, which as known, is used to control the operation of the computing unit by controlling the execution of other programs, controlling communication with peripheral devices and controlling use of the computer resources.

Central processing unit 810 is coupled to main storage 820, which is directly addressable and provides for high-speed processing of data by the central processing unit. Main storage may be either physically integrated with the CPU or constructed in stand-alone units.

Main storage 820 is also coupled to one or more input/output devices 830. These devices include, for instance, keyboards, communications controllers, teleprocessing devices, printers, magnetic storage media (e.g., tape, disk), direct access storage devices, and sensor-based equipment. Data is transferred from main storage 820 to input/output devices 830, and from the input/output devices back to main storage.

The present invention can be included in an article of manufacture (e.g., one or more computer program products) having for instance, computer usable media. The media has embodied therein, for instance, computer readable program code means for providing and facilitating the capabilities of the present invention. The articles of manufacture can be included as part of a computer system or sold separately. Additionally, at least one program storage device readable by a machine, tangibly embodying at least one program of instructions executable by the machine to perform the capabilities of the present invention can be provided.

The flow diagrams depicted herein are just exemplary. There may be many variations to these diagrams or the steps (or operations) described therein without departing from the spirit of the invention. For instance, the steps may be performed in a differing order, or steps may be added, deleted or modified. All of these variations are considered a part of the claimed invention.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined by the following claims.

What is claimed is:

1. A method of assessing a combinatorial library for complementarity to a target having at least one binding site, said combinatorial library comprising a plurality of ligands, each based on a common core, said method comprising:
docking each ligand of the plurality of ligands to the target molecule to generate a plurality of ligand positions relative to the target molecule in a plurality of ligand-target molecule complex formations, said plurality of ligand positions comprising a plurality of common core positions relative to the target molecule;
determining an rms deviation of each common core position of said plurality of common core positions from other common core positions; and
forming clusters according to said rms deviation.

2. A method according to claim 1, additionally comprising rating complementarity of the combinatorial library to the target molecule according to number of ligands in a cluster having a minimum rms deviation relative to number of ligands in the combinatorial library.

3. A method according to claim 1 wherein said determining an rms deviation comprises:
placing a grid around a binding site of the target molecule;
for each ligand position, determining a location on the grid corresponding to the center of mass of the common core; and
determining the rms deviation of each common core position from every other common core position having a location on the grid within a predetermined distance.

4. A method according to claim 1 wherein said forming clusters comprises forming clusters using a single linkage clustering algorithm.

5. A method according to claim 1 wherein said docking each ligand comprises:
performing a pre-docking conformational search to generate multiple solution conformations of each ligand;
generating a binding site image of the target molecule, said binding site image comprising multiple hot spots;
matching hot spots of the binding site image to atoms in at least one solution conformation of the multiple solution conformations of each ligand to obtain at least one ligand position relative to the target molecule in a ligand-target molecule complex formation; and
optimizing the at least one ligand position while allowing translation, orientation and rotatable bonds of the ligand to vary, and while holding the target molecule fixed.

6. A system for assessing a combinatorial library for complementarity to a target having at least one binding site, said combinatorial library comprising a plurality of ligands, each based on a common core, said system comprising:
means for docking each ligand of the plurality of ligands to the target molecule to generate a plurality of ligand positions relative to the target molecule in a plurality of ligand-target molecule complex formations, said plurality of ligand positions comprising a plurality of common core positions relative to the target molecule;
means for determining an rms deviation of each common core position of said plurality of common core positions from other common core positions; and
means for forming clusters according to said rms deviation.

7. A system according to claim 1, additionally comprising means for rating complementarity of the combinatorial library to the target molecule according to number of ligands in a cluster having a minimum rms deviation relative to number of ligands in the combinatorial library.

8. A system according to claim 1 wherein said means for determining an rms deviation comprises:
means for placing a grid around a binding site of the target molecule;
means for, for each ligand position, determining a location on the grid corresponding to the center of mass of the common core; and
means for determining the rms deviation of each common core position from every other common core position having a location on the grid within a predetermined distance.

9. A system according to claim 1 wherein said means for forming clusters comprises means for forming clusters using a single linkage clustering algorithm.

10. A system according to claim 1 wherein said means for docking each ligand comprises:
means for performing a pre-docking conformational search to generate multiple solution conformations of each ligand;
means for generating a binding site image of the target molecule, said binding site image comprising multiple hot spots;
means for matching hot spots of the binding site image to atoms in at least one solution conformation of the multiple solution conformations of each ligand to obtain at least one ligand position relative to the target molecule in a ligand-target molecule complex formation; and
means for optimizing the at least one ligand position while allowing translation, orientation and rotatable bonds of the ligand to vary, and while holding the target molecule fixed.

11. At least one program storage device readable by a machine, tangibly embodying at least one program of instructions executable by the machine to perform a method for assessing a combinatorial library for complementarity to a target having at least one binding site, said combinatorial library comprising a plurality of ligands, each based on a common core, said method comprising:
docking each ligand of the plurality of ligands to the target molecule to generate a plurality of ligand positions relative to the target molecule in a plurality of ligand-target molecule complex formations, said plurality of ligand positions comprising a plurality of common core positions relative to the target molecule;
determining an rms deviation of each common core position of said plurality of common core positions from other common core positions; and
forming clusters according to said rms deviation.

12. The at least one program storage device according to claim 1, wherein said method additionally comprises rating complementarity of the combinatorial library to the target molecule according to number of ligands in a cluster having a minimum rms deviation relative to number of ligands in the combinatorial library.

13. The at least one program storage device according to claim 1, wherein said determining an rms deviation comprises:
placing a grid around a binding site of the target molecule;
for each ligand position, determining a location on the grid corresponding to the center of mass of the common core; and
determining the rms deviation of each common core position from every other common core position having a location on the grid within a predetermined distance.

14. The at least one program storage device according to claim 1, wherein said forming clusters comprises forming clusters using a single linkage clustering algorithm.

15. The at least one program storage device according to claim 1, wherein said docking each ligand comprises:
performing a pre-docking conformational search to generate multiple solution conformations of each ligand;
generating a binding site image of the target molecule, said binding site image comprising multiple hot spots;
matching hot spots of the binding site image to atoms in at least one solution conformation of the multiple solution conformations of each ligand to obtain at least one ligand position relative to the target molecule in a ligand-target molecule complex formation; and
optimizing the at least one ligand position while allowing translation, orientation and rotatable bonds of the ligand to vary, and while holding the target molecule fixed.
